(19) United States
(12) Patent Application Publication
(10) Pub. No.: US 2024/0016611 A1
(43) Pub. Date: Jan. 18, 2024

(54) DEVICE AND METHOD TO ASSIST REVISION SURGERIES FOR KNEE ARTHROPLASTY

(71) Applicants: Mary Hitchcock Memorial Hospital, for itself and on behalf of Dartmouth-Hitchcock Clinic, Lebanon, NH (US); The Trustees of Dartmouth College, Hanover, NH (US)

(72) Inventors: Rebecca Thomson, Hopkinton, NH (US); Peder Solberg, Hanover, NH (US); Douglas Van Citters, Hanover, NH (US); Wayne Moschetti, Hanover, NH (US); Alexander Orem, Norwich, VT (US)

(21) Appl. No.: 18/222,275

(22) Filed: Jul. 14, 2023

Related U.S. Application Data
(60) Provisional application No. 63/390,155, filed on Jul. 18, 2022.

(51) Int. Cl. A61F 2/30; A61F 2/38

(52) U.S. Cl. CPC A61F 2/30734; A61F 2/3859; A61F 2002/30561; A61F 2002/30408; A61F 2002/30556; A61F 2002/30736; A61F 2002/3895

(57) ABSTRACT

A reference-point providing (RPP) device for conversion of unicompartmental knee arthroplasty (UKA) to total knee arthroplasty (TKA), has a wedge having a joint surface, a bottom flat surface, and a second surface, the second surface extending from the bottom flat surface to the joint surface at a thick end of the wedge and a mounting flange; the wedge fitting in a space remaining after removal of a UKA implant with the joint surface providing a reference point for determining alignment of a TKA implant. Converting a UKA to TKA with the RPP requires removing UKA implant from a femur of a patient, inserting the RPP and attaching it to the femur; using a posterior reference guide having a first paddle on a condyle of the femur and a second paddle on the RPP, to determine necessary alignment and rotation of the TKA implant; and removing the RPP.

### CROSS-REFERENCE TO RELATED APPLICATIONS

This application benefits from and claims priority to U.S. Provisional Patent Application Ser. No. 63/390,155, filed Jul. 18, 2022. The entire disclosure of the aforementioned application is incorporated by reference herein as if fully set forth.

### BACKGROUND

Knee arthroplasty has become common because of the high incidence of osteoarthritis among the elderly, as well as injury-induced knee degradations, among other indications, and the improved availability of medical care. Typically, knee arthroplasty surgery may take one of two forms, a unicompartmental form (partial or unicompartmental knee arthroplasty, here referred to as UKA), FIG. 1A, where only one side of the articular surface of femur and tibia are replaced by prosthetic implants, and a bicompartmental form (total knee arthroplasty, here referred to as TKA) FIG. 1B, where prosthetic implants replace the full articular surface of the distal end of the femur and proximal end of the tibia, typically with mechanical links supplementing the ligaments that hold femur and tibia together in the normal knee.

UKA is done in about 8% of knee arthroplasty surgeries and is preferred by many patients where only part of their natural knee joint has been damaged because pain and recovery time are often less than those encountered with total knee arthroplasty.

On occasion, patients who have undergone UKA develop further arthritic symptoms or suffer additional joint damage and require further surgery to convert the UKA to TKA. In this conversion, the unicompartmental implants must be removed, additional bone removed, and new TKA implants that replace the entire articular surface of both the distal end of femur and proximal end of tibia inserted.

We note the prosthetic femur implant of UKA differs in shape from, and is less than half the width of, femur implants of TKA.

It is desirable to correctly align total knee implants when performing knee arthroplasty, both initially and, where revision surgery is required, during revisions.

Normal knees provide hinge joints having a same axis of rotation in both legs. Incorrectly aligned or rotated TKA implants result in a hinge joint with an abnormal axis of rotation twisted with respect to the normal axis of rotation of the knee. This abnormal axis of rotation requires sometimes-painful compensation at hip and ankle and can result in significant gait disturbances, ongoing pain, excessive stress on remaining ligaments that help stabilize the joint, chondromalacia patella due to incorrect alignment of the patellar tendon and patella over the knee joint, and other issues we can refer to as “poor patient outcomes.”

### SUMMARY

In an embodiment, a reference-point providing (RPP) device for use in conversion of unicompartmental knee arthroplasty (UKA) to total knee arthroplasty (TKA), has a wedge having a joint surface, a bottom flat surface, and a second surface, the second surface extending from the bottom flat surface to the joint surface at a thick end of the wedge; a mounting flange coupled to the wedge; and the wedge is configured to fit in a space remaining after removal of a UKA implant with the joint surface providing a reference point for determining alignment of a TKA implant.

In another embodiment, the RPP is used to convert a unicompartmental knee arthroplasty (UKA) to total knee arthroplasty (TKA), by removing a previously-implanted UKA implant from a femur of a patient, inserting the RPP and pinning or screwing it to the femur of the patient; using a posterior reference guide having a first paddle on a first reference point on a condyle of the femur of the patient and a second paddle on a second reference point of the RPP, to determine necessary alignment and rotation of the TKA implant; and removing the RPP.

### BRIEF DESCRIPTION OF THE FIGURES

FIG. 1A is an illustration of knee joint bones post UKA, as known in the art.

FIG. 1B is an illustration of a knee joint bones post TKA, as known in the art.

FIG. 2 is a lateral view of a device for providing a reference point at a level of a natural femur surface for use with a TKA implant positioning guide when the femur surface has previously been cut away for an UKA implant, in embodiments.

FIG. 3 is a top view of the device of FIG. 2.

FIG. 4 is an end view of the device of FIG. 2.

FIGS. 5 and 6 are perspective views of the device of FIG. 2 rotated to better show how the device for providing a reference point is shaped.

FIG. 7A is a photograph of a model of femur and tibia with the device for providing a reference point screwed to the femur.

FIG. 7B is a photograph of a model of femur with the device for providing a reference point screwed to the femur, the snap-off handle removed, and a posterior alignment guide in place.

FIGS. 8A, 8B, and 8C are closeups of a breakaway joint between the snap-off handle and the arcuate wedge of the device for providing a reference point, each figure showing the joint under two levels of magnification.

FIGS. 9A, 9B, and 9C are an end view, a perspective view, and a lateral view of an alternative embodiment of the device for providing a reference point having a slot for attachment to an installation tool in place of a snap-off handle.

FIGS. 10A, 10B, and 10C are an end view, a perspective view, and a lateral view respectively of an alternative embodiment of the device for providing a reference point having a pin hole for attachment to an installation rod in place of a snap-off handle; the rod is illustrated in FIG. 10D.

FIG. 11 is flowchart illustrating use of the device in performing UKA to TKA conversion surgeries.

FIGS. 12A and 12B illustrate an embodiment where thickness and pitch of the wedge is continuously adjustable by rotation of adjustment wheels.

FIG. 13 illustrates an embodiment where thickness of the wedge is adjustable in discrete increments by addition of pegged shims.

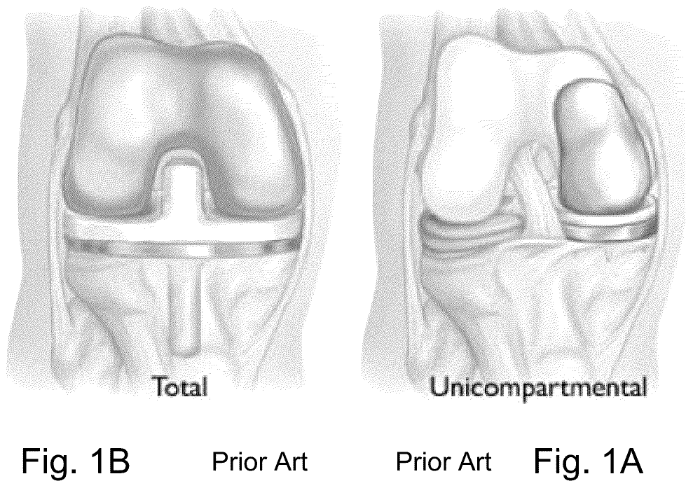

### DETAILED DESCRIPTION OF THE EMBODIMENTS

In standard TKA surgery, rotation of the implant about the long axis of the femur is set by aligning two paddles on a posterior reference guide to a reference point on each of the medial and lateral posterior femoral condyles, which provide two, a medial and a lateral, necessary reference points for setting the alignment. Unfortunately, UKA surgery typically removes significant portions of either the medial or lateral femoral condyle to make room for the unilateral implant. For patients undergoing UKA revision to TKA surgery, their unilateral UKA implant must be removed to adequately prepare the bone for a new prosthesis or implant. When the UKA implant is removed, a void remains on the femoral condyle where the bone had been removed at the time of the UKA procedure. The paddles of the traditional positioning guide thus cannot be used to establish the appropriate femoral rotation because the femoral condyle no longer has its natural, original, shape, leaving a surgical cavity including a gap between remaining bone and usual reference point on one of the medial or lateral femoral condyles.

Thus, to set the rotation of the new TKA femoral component, surgeons often visually guess a correct alignment of the posterior reference paddle on the side where the UKA implant had been because that reference point no longer exists on the bone. This can lead to their inaccurately setting rotation leading to malposition of the femoral component and poor patient outcomes.

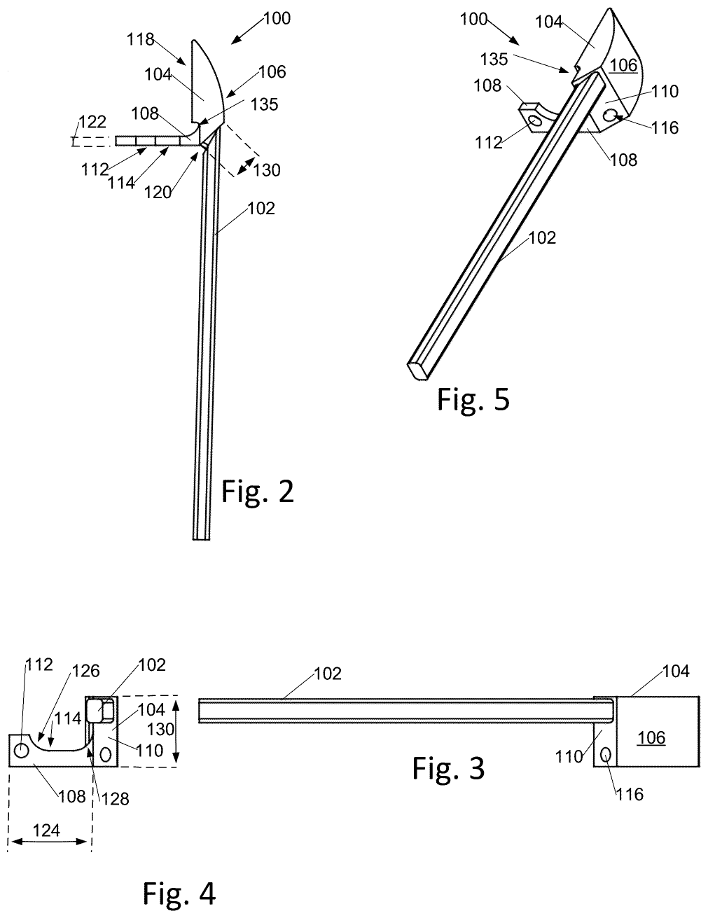

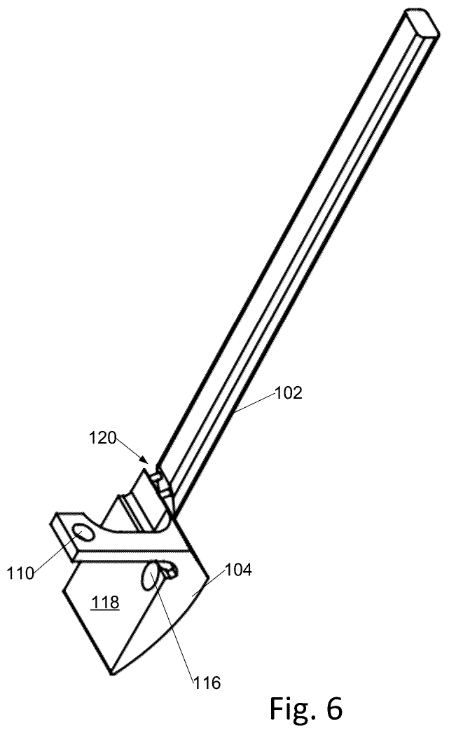

We developed a reference point providing device 100 (RPP), illustrated in FIGS. 2, 3, 4, 5, and 6, that is used after removal of the UKA implant, which fits into the surgical cavity where the UKA implant had been, and which provides a reference point for a positioning guide to replace the reference point that no longer exists on the bone.

This reference-point providing device 100 is formed of 3-D printed plastic and has a snap-off handle 102 coupled to an arcuate wedge 104 with a curved surface 106 and an angled rear surface 110 that in some embodiments is flat; the curved surface being a joint surface configured to substitute for the curved joint surface of the femur that was removed in unicompartmental knee arthroplasty and which, in normal knees, rides on cartilage over the proximal end of the tibia. While surface 106 is curved as preferred in many situations, hence the name arcuate wedge, in some embodiments curved surface 106 may be flat and have arcuate wedge 104 is replaced by a wedge having trapezoidal or triangular prism shape; in these embodiments the joint surface may be flat. Also coupled to arcuate wedge 104 near the angled rear surface 110 is a mounting flange 108. Mounting flange 108 has an optional hole 112 of diameter 3.5 mm adapted for installation of a pin or screw to hold reference-point providing device 100 in place on a femur during use, and an arcuate cutout 114 that has a first 126 and second 128 curved edge of cutout 114 with diameter of curve 5 mm. In embodiments, angled rear surface 110 has a second optional hole 116 which in embodiments has diameter 3.5 mm and is adapted for installation of a pin or screw, second hole 116 penetrates to a bottom flat surface 118 of arcuate wedge 104. Attachment of snap-off handle 102 to arcuate wedge 104 has a breakaway joint 120 with notches to concentrate stress and direct breakage during a snapping-off of snap-off handle 102 from arcuate wedge 104. Arcuate wedge 104 has width between ten and mm, and angled rear surface 110 has length 130 of 8.5 mm, for example.

A thickness 122 of mounting flange 108 is, in an embodiment, 2.25 millimeters (mm), but in other embodiments may range from 2 to 3 mm, mounting flange 108 typically has length 124 of 21 mm.

The arcuate wedge 104 has a curved surface 106, a bottom flat surface 118, and a second or angled flat surface 110, the angled rear surface 110 that in some embodiments extends from the bottom flat surface 118 to the curved surface 106 at a thick end of the arcuate wedge. In some embodiments, bottom flat surface 118 is notched 135 near mounting flange 108, in which case the angled rear surface 110 extends from notch 135 to the arcuate surface 106.

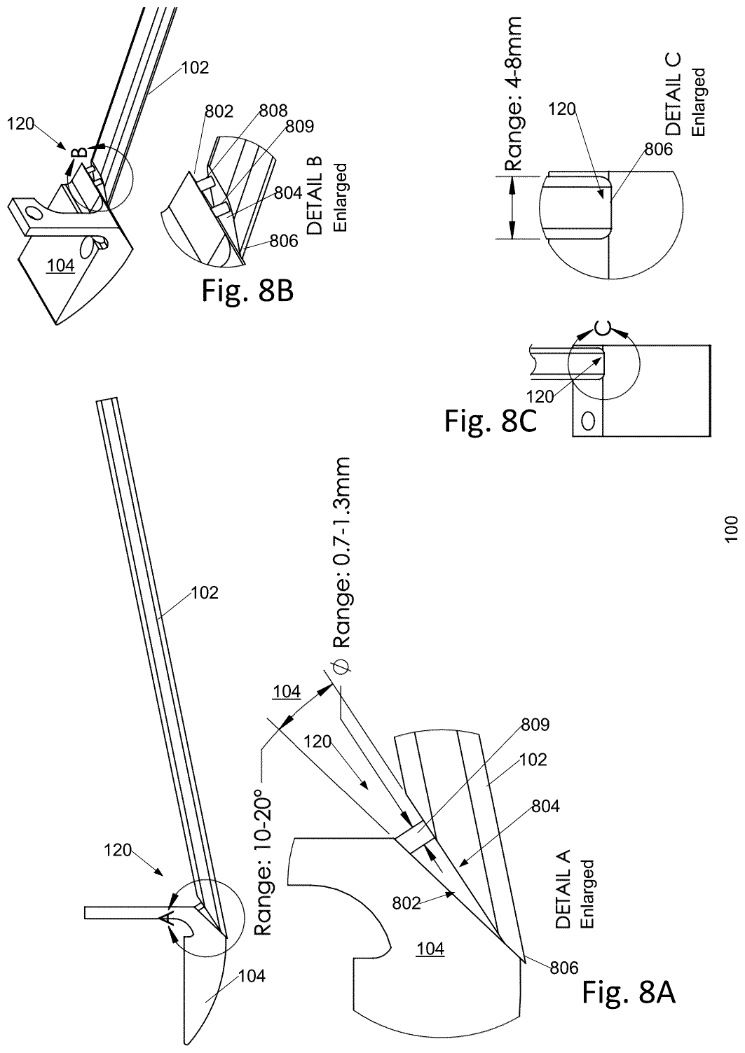

The breakaway joint 120 between snap-off handle 102 and arcuate wedge 104 is illustrated in more detail in FIGS. 8A, 8B, and 8C. The arcuate wedge 104 has a flat surface 802 extending from a thin molded hinge 806, and snap-off handle 102 has a flat surface 804, extending from the thin molded hinge 806. Bridging the gap between flat surface 802 and flat surface are a pair of coupling pegs 808, 809. Coupling pegs 808, 809 and thin molded hinge 806 are configured such that, if arcuate wedge 104 is retained in position, and snap-off handle 102 is moved in two axes, coupling pegs 808, 809 will fracture as will thin molded hinge 806, thereby permitting removal of snap-off handle 102 from arcuate wedge 104.

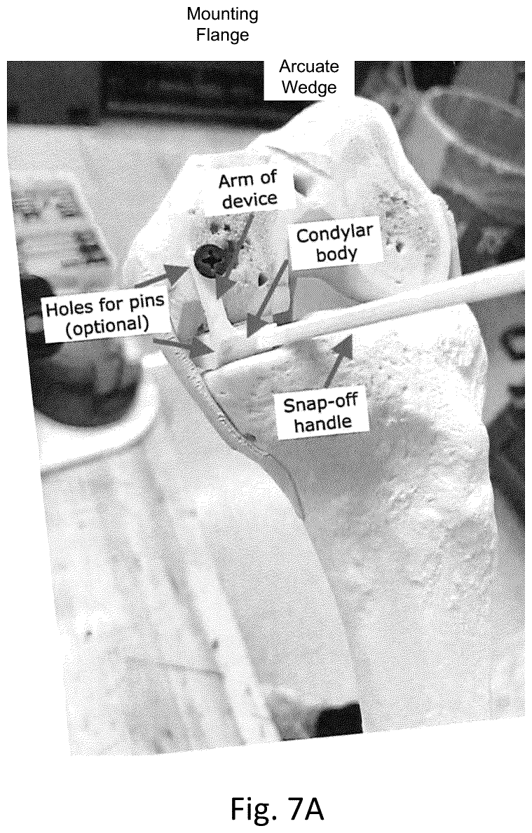

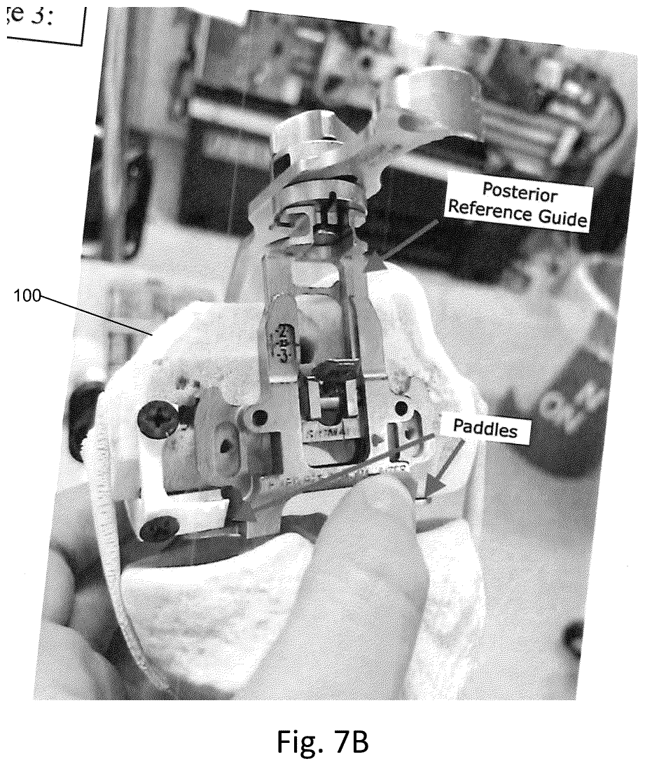

The reference point providing device 100 facilitates accurate and precise rotational alignment of the femoral component in a UKA to TKA conversion surgery. The reference point providing device 100 inserts into the aforementioned void or surgical cavity as illustrated in FIGS. 7A and 7B; and is described in further detail below.

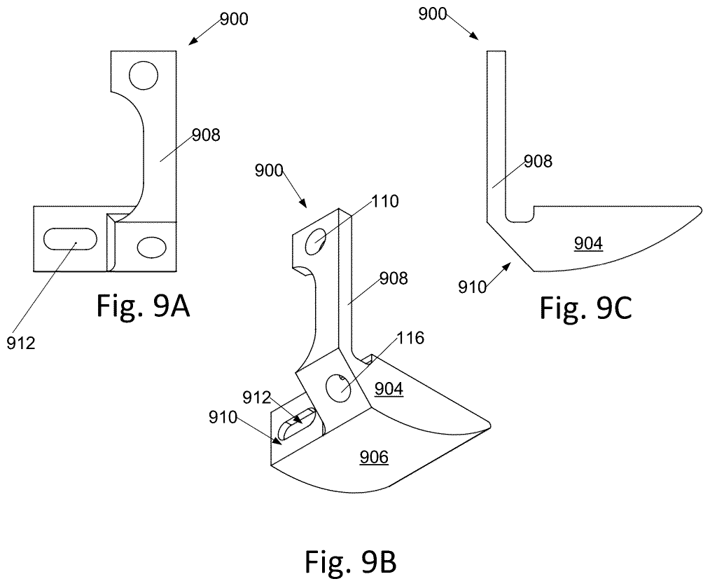

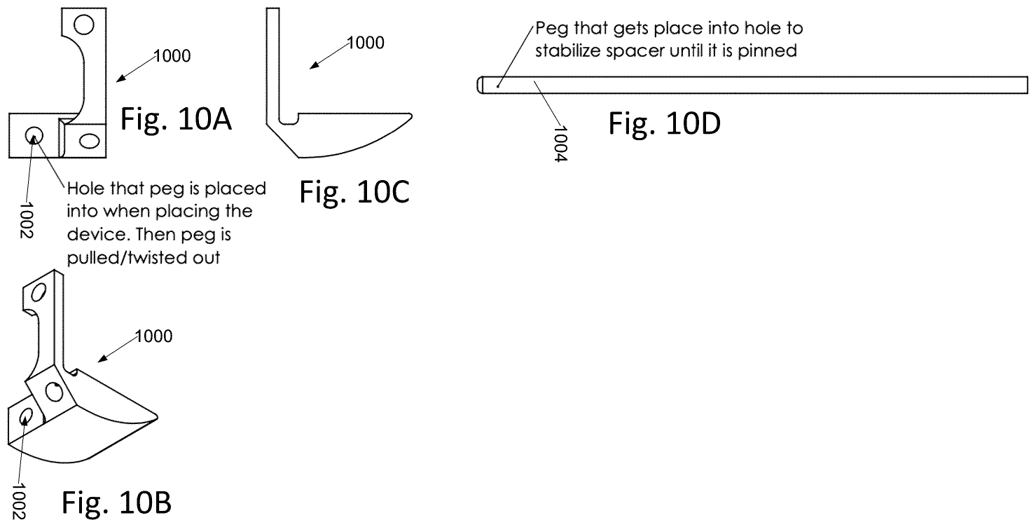

In an alternative embodiment 900, as illustrated in FIGS. 9A, 9B, and 9C, the arcuate wedge 904 has a curved surface 906 and an angled rear surface 910 that in some embodiments is flat. Also coupled to arcuate wedge 904 near the angled rear surface 910 is a mounting flange 908; this shape resembles arcuate wedge 104 of the embodiment of FIG. 2, however there is no snap-off handle 102. In place of snap-off handle 102, arcuate wedge 904 has an elongated slot 912 adapted for coupling to an insertion tool such as a modified forceps. In another alternative embodiment 1000 illustrated in FIGS. 10A, 10B, 10C, and 10D, a peg hole 1002 is provided instead of slot 912 along with a peg-shaped handle 1004 configured to friction-fit in peg hole 1002, in alternative embodiments peg-shaped handle 1004 is threaded into peg hole 1002. Once the device is inserted and pegged to the femur, the peg-shaped handle can be removed by twisting or pulling the peg-shaped handle from peg hole 1002 or unscrewing the peg-shaped handle 1004 from peg hole 1002 in embodiments where peg-shaped handle 1004 is threaded into peg hole 1002.

We note that the embodiments of FIG. 2 through FIG. 10D are manufactured in right and left chiral forms. Further, thickness of arcuate wedge 104, 904 varies with the manufacturer's make and model of the removed UKA prosthetic and ranges from 4-12 mm.

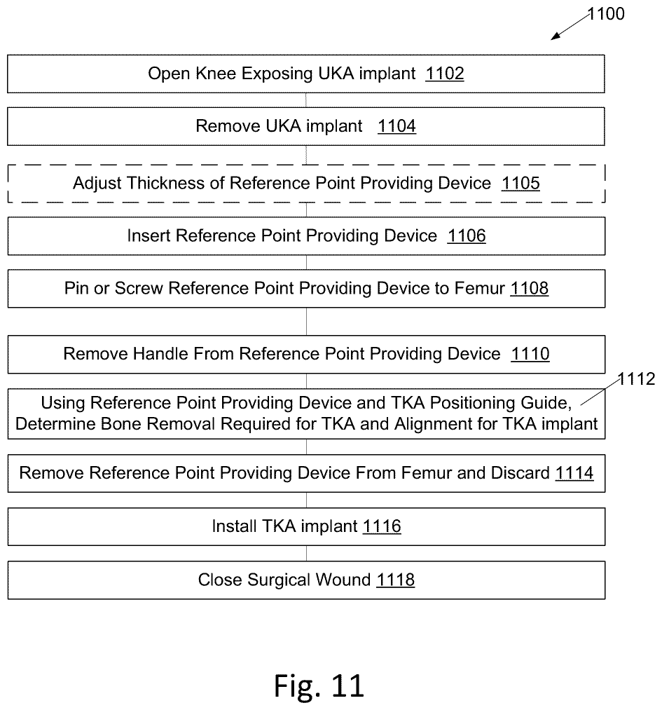

The reference point providing device is used according to a method 1100 (FIG. 11) of revising UKA to TKA that includes opening the knee 1102, removing the previously-implanted UKA implant 1104, and inserting 1106 the reference point providing device of FIG. 2, 9A-9B-9C, or 10A-10B-10C into the joint space formerly occupied by the UKA implant. In embodiments where thickness of the reference point providing device is adjustable, thickness of the arcuate wedge of the reference point providing device is optionally adjusted 1105 prior to insertion into the jointspace. The reference point providing device is then pinned or screwed 1108 to the bone to hold it stable while bone is trimmed. The device handle is then removed 1110 from the reference pointing device of FIG. 2, the peg-shaped handle from the device of FIGS. 10A, 10B, and 10C, or the insertion tool from the device of FIGS. 9A, 9B, and 9C.

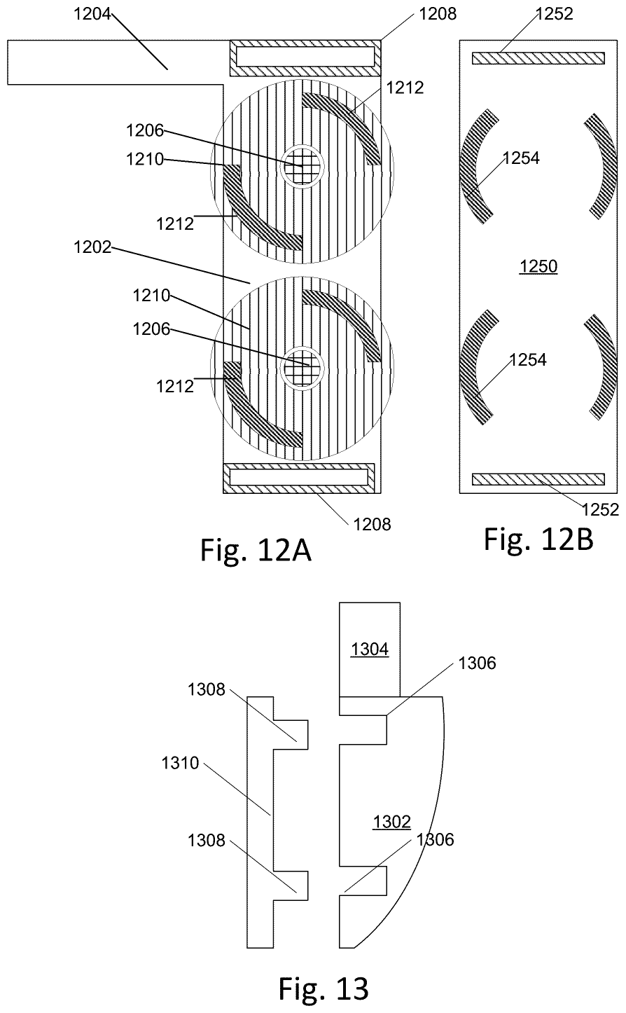

Embodiments with wedges, which may be arcuate wedges as previously described, of adjustable thickness are illustrated in FIGS. 12A-12B and 13.

FIG. 12A illustrates a body 1202 of a reference point providing device with handle 1204 having molded onto the body 1202 a pair of pins 1206. Also molded onto device body 1202 are a pair of raised rectangular slotted guides 1208. Over the pins 1206 is placed an adjustment wheel 1210 having molded onto it two (or in some embodiments three or four) semicircular wedges 1212. FIG. 12B illustrates a cap piece 1250 for the reference point device body 1202 that is inverted and placed over the reference point device body 1202. This cap piece 1250 has a pair of raised ridges 1252 molded into it that interface with slotted guides 1208 of device body, and semicircular wedges 1254 configured so, with cap piece 1250 positioned with raised ridges 1252 inserted into slotted guides 1208, the semicircular wedges 1254 engage with semicircular wedges 1212 of the adjustment wheels 1210. This provides an adjustable thickness of the overall reference point providing device wedge that changes with rotation of the adjustment wheels 1210. Adjustment is performed by rotating adjustment wheels 1210 to an appropriate setting.

In the embodiment of FIG. 13, the reference point providing device has an arcuate wedge body 1302 to which handle 1304 is attached, the wedge body having holes 1306 provided for pins 1308 that are attached to removable shims 1310. With this embodiment, adjustment of thickness is performed by selecting zero, one, or more shims of appropriate thickness and inserting pins 1308 of the shims into holes 1306 of the wedge body.

The femoral condyle that was not cut during the UKA provides one reference point for a posterior reference guide, and the reference point providing device then provides the second reference point necessary for alignment of the posterior reference guide's paddles. The body of the device mimics the condylar geometry to the extent necessary to complete the alignment accurately and successfully. Once a posterior reference guide has been positioned and used, additional bone is removed from the femoral condyle that was not cut during the UKA surgery to, with space provided by removal of the reference point providing device, provide space for a TKA implant.

Mounting flange 108 extends from the condylar body or arcuate wedge 104 of the device and references the distal femoral cut plane which sets the correct superior-inferior position of the reference point providing device and prevents it from rotating about the femur's anteroposterior axis while cuts are made for the opposite side of the TKA implant, and the posterior alignment guide is used to determine 1112 proper alignment and rotation of the TKA implant that will shortly be installed. With both the reference point providing device attached to the femur and its handle removed, and the posterior reference guide in place, the femur device, and guide will be positioned as in FIG. 7B. Curved surface 106 of arcuate wedge provides a curve similar to that of a natural femur and provides the reference point needed for the posterior reference guide that is missing because of prior UKA.

The pegs or screws holding the reference point providing device of FIG. 2, 9A-9B-9C, or 10A-10B-10C to the femur are then removed and the reference point providing device is removed 1114 and, since it is disposable, discarded in most embodiments. The remainder of the TKA conversion proceeds as normal with removal of any additional bone required to fit the TKA implant, installation of the TKA implant 1116, and closure 1118 of the surgical wound.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A reference-point providing (RPP) device for use in conversion of unicompartmental knee arthroplasty (UKA) to total knee arthroplasty (TKA), comprising:
a wedge having a joint surface, a bottom flat surface, and a second surface, the second surface extending from the bottom flat surface to the joint surface at a thick end of the wedge;
a mounting flange coupled to the wedge; and
wherein the wedge is configured to fit in a space remaining after removal of a UKA implant with the joint surface providing a reference point for determining alignment of a TKA implant.

2. The RPP of claim 1, further comprising a snap-off handle coupled to the wedge.

3. The RPP of claim 1, wherein a peg hole is formed in the wedge for attachment of a peg handle.

4. The RPP of claim 3, where the peg hole is threaded.

5. The RPP of claim 4 further comprising a handle threaded into the peg hole.

6. The RPP of claim 3, further comprising a peg-shaped handle friction-fit in the peg hole.

7. The RPP of claim 2 wherein the joint surface of the wedge of the RPP is an arcuate surface.

8. The RPP of claim 7 where the wedge has adjustable thickness.

9. The RPP of claim 3 where the wedge is an arcuate wedge.

10. The RPP of claim 9 where the wedge has an abustable thickness.

11. The RPP of claim 2 where the mounting flange coupled to the wedge has at least one hole for accepting a mounting pin or screw.

12. The RPP of claim 3 where the mounting flange coupled to the wedge has at least one hole for accepting a mounting pin or screw.

13. The RPP of claim 2 wherein a second hole is formed in the wedge between the second flat surface and the bottom flat surface to accept a mounting pin or screw.

14. The RPP of claim 3 wherein a second hole is formed in the wedge between the second flat surface and the bottom flat surface to accept a mounting pin or screw.

15. A method of converting a unicompartmental knee arthroplasty (UKA) to total knee arthroplasty (TKA), comprising:
removing a previously-implanted UKA implant from a femur of a patient, inserting a reference point providing (RPP) device comprising:
a wedge having a joint surface, a bottom flat surface, and a second surface, the second surface extending from the bottom flat surface to the joint surface at a thick end of the wedge;
a mounting flange coupled to the wedge; and
wherein the wedge is configured to fit in a space remaining after removal of a UKA implant with the joint surface providing a reference point for determining alignment of a TKA implant;
pinning or screwing the RPP device to the femur of the patient;
using a posterior reference guide having a first paddle on a first reference point on a condyle of the femur of the patient and a second paddle on a second reference point of the RPP, to determine necessary alignment and rotation of the TKA implant; and
removing the RPP.

16. The method of claim 15 further comprising removing a handle from the RPP device.

17. The method of claim 16 where removing the handle comprises breaking off a snap-off handle.

18. The method of claim 16 where removing the handle comprises removing the handle from a hole in the RPP device.

19. The method of claim 15 where the joint surface of the wedge has an arcuate shape.
